(19) United States
(12) Patent Application Publication
(10) Pub. No.: US 2013/0230576 A1
(43) Pub. Date: Sep. 5, 2013

(54) MICROLAYER COEXTRUSION TO CREATE A TIME-RELEASE DRUG

(71) Applicant: GUILL TOOL & ENGINEERING, West Warwick, RI (US)

(72) Inventors: Richard R. Guillemette, West Warwick, RI (US); Christopher J. Hummel, West Warwick, RI (US); Robert G. Peters, Providence, RI (US)

(73) Assignee: GUILL TOOL & ENGINEERING, West Warwick, RI (US)

(21) Appl. No.: 13/681,413

(22) Filed: Nov. 19, 2012

Related U.S. Application Data
(60) Provisional application No. 61/561,165, filed on Nov. 17, 2011.

(51) Int. Cl. A61K 9/00

(52) U.S. Cl. CPC A61K 9/0002
USPC 424/423; 424/400; 424/464; 424/451

(57) ABSTRACT

Medical devices containing time release drug substance are disclosed, including medical tubing, catheters, stents, cables (including fiber optic cables), pills, capsules, sheaths, threads, clamps, sutures, and endotracheal devices. A method for extruding multiple laminated flow streams using microlayer coextrusion to create various time release drug delivery products is also disclosed.

### CROSS-REFERENCE TO RELATED APPLICATIONS

This application claims priority to and the benefit of U.S. Provisional Application Ser. No. 61/561,165, filed on 17 Nov. 2011, the disclosure of which is incorporated herein by reference in its entirety.

### FIELD

The present disclosure relates to medical devices containing time-release drug substance, and more particularly, to medical tubing, catheters, stents, cables (including fiber optic cables), pills, capsules, sheaths, implants, threads, clamps, sutures, tapes, sheets and endotracheal devices. The present disclosure also generally relates to a method for extruding multiple laminated flow streams using microlayer coextrusion to create these various time-release drug delivery products.

### BACKGROUND

The use of polymers in biomedical applications has been on a rise since they were first introduced in this field. This has been possible due to the unique combination of properties exhibited by polymers such as flexibility, ease of processing and excellent biocompatibility. Biopolymers are being used in many medical devices involving life saving applications. Artificial implants, drug delivery systems, lubricious coatings for less invasive devices, biological adhesives, anti-thrombogenic coatings and soft tissue replacements are a few of the current commercial applications. Researchers around the world are trying to improve these materials to make them more versatile in their applications with an aim to eliminate the current problems associated with them.

### SUMMARY

The disclosed embodiments relate to an extruded medical device capable of delivering an agent in a time dependent fashion. In a microlayer extrusion process, each of a laminated flow stream(s) (containing the desired agent(s)) is subject to repeated steps in which the flows are divided and overlapped to amplify the number of laminations. The amplified laminated flows are rejoined to form a cumulated laminated output which may achieve dimensions as thin as the micro or nanometer range.

The aspects of the present disclosure relate to an extruded medical device comprising one or more pharmaceutical product(s) or drug substances (including mixtures thereof) layered with one or more biocompatible materials that control the time release of the delivery of the drug substance.

Medical devices include catheters, stents, threads, cables (including fiber optic cables), pills, capsules, lozenges and tablets.

Biocompatible materials include polymers such as polyamides, polyimides, polyureas and poly(urethane-urea)s, MPC polymer, polyesters, polyethers, etc. Polyamides such as Polyether Block Copolyamides (PEBA), medical grade polyamide 11, and MED polyamide are particularly useful polymers. Nanocellulose fibers are additional biocompatible materials.

One embodiment relates to a medical tubular device comprising: a polymeric tube containing nano or micro-sized features and a drug substance.

Another embodiment relates to a medical tubular device wherein said medical tubular device is a stent. Another embodiment of said stent contains nano or micro-sized features formed into folds or skin layers. Another embodiment of said stent containing nano or micro-sized features is formed into folds or skin layers that have separated at the fold interface.

Another embodiment relates to a medical tubular device comprising: a multi-component polymeric tube containing an embedded stem of a first polymer and a support surface surrounding said embedded stem containing a second polymer wherein each of said first and second polymer may contain one or more drug substances.

Another embodiment relates to a medical tubular device as described above wherein said medical tubular device is an orally administrable medicament such as a pill or capsule.

Another embodiment relates to a medical device comprising a polymer solid of annular rings emanating from the center of a drug substance core. Said polymer solid may contain from ten to thousands of annular layers.

Another embodiment relates to a medical device as described above wherein said device is a pill or capsule comprising a polymer solid of annular rings emanating from the center of a drug substance core.

Another embodiment relates to a medical device comprising a polymer solid of annular rings containing a drug substance emanating from a center inactive core.

In another embodiment, each layer of the medical device may be comprised of one or more pharmaceutical product(s) or drug substances (including mixtures thereof) alternating with one or more materials that control the time release of the delivery of the drug substance.

Although the aspects of the present disclosure are generally directed to drug delivery, the aspects of the disclosed embodiments are not so limited and may include any product, composition or substance, for which time release properties are desirable. These may include for example, but are not limited to, vitamins, medicaments, active and non-active ingredients.

Suitable pharmaceutical agents include antibiotics, angiogenics (such as Fibroblast growth factor (FGF), VEGF, and angiopoietins such as Ang1 and Ang2), anti-angiogentics (such as bevacizumab, thalidomide, itraconazole, carboxyamidotriazoles, angiostatin, endostatin, linomide), immunomodulators (such as immunophilins ciclosporins, rapamycins, sirolimus, zotarolimus, everolimus, glucocorticoids, cytostatics such as alkylating agents, antimetabolites), anti-inflammatories (such as salicylates, COX-2 inhibitors, propionic acid derivatives, acetic acid derivatives, enolic acid (oxicam) derivatives, fenamic acid derivatives (fenamates) and sulphonanilides), antithrombotics (such as warfarins, heparins and Factor Xa inhibitors), platelet aggregation inhibitors (such as ticlopidine or clopidogrel), antiproliferatives (such as Paclitaxel).

Suitable devices include a drug substance at, for example, concentrations of between 1-99 percent dry weight, more particularly 35-80 percent dry weight. Such devices may also elute the drug substance for extended periods. For example some products may elute drug substance for up to 30-60 days.

In one embodiment, the cumulated laminated output may consist solely of drug substances, with each layer containing a different drug substance. The layers may be alternated in any suitable fashion. When the cumulated laminated output includes time-release components or layers, as each time-release layer is dissolved by the body, a layer of drug substance would be administered in a manner generally understood in the art.

Some of the aspects of the disclosed embodiments are directed to forming the layers resulting in the cumulated laminated output by a microlayer coextrusion die. The microlayer coextrusion die forms the layers in annular rings that emanate from the center of the drug substance delivery. The layers that form the drug substance containing device are concentric. The drug substance containing polymer may be extruded in various cross-sections, such as circular, elliptical, or oval shapes. The thickness of each layer may also vary depending upon one or more factors such as the desired time release or the required dosage of the drug. The nanolayer die is used to make the product, except that the center is not hollow.

The time release characteristics of the produced drug delivery device may be controlled through the barrier properties of the annular rings. Small micro- or nano-sized layers may induce confined polymer crystallization. These confined crystals may result in unique barrier properties. Polymer nanocomposities may also be used to control the properties of the drug substance. Particles added to the polymer may alter the diffusivity of the polymer surface or change crystal orientation. These particles may also affect barrier properties by providing a tortuous path for a permeate to travel. Permeation though the multilayer structure may be enhanced or impeded through layer size or the introduction of particles including nanoparticles.

In a reservoir drug delivery system, a concentrated drug substance core is surrounded by a permeable membrane. This membrane may be comprised of nondegradable or biodegradable polymers. The diffusivity of the membrane may be tailored using microlayer coextrusion. The annular rings created by the micro- or nano-layer die form a membrane composed of multiple polymer layers where the diffusivity is determined by the polymers used and also the layer properties created through microextrusion. In a biodegradable membrane the erosion of the membrane may also be controlled through the microlayered annular rings. In typical biodegradable systems, as the polymer degrades the surface area of the drug substance increases. This may lead to a varying drug release rate. With a microlayered time-release drug substance the thickness and concentration may be altered to achieve a more uniform and constant release. Drug substance microparticles (and/or nano particles) may also be added to individual or multiple layers. As the layers biodegrade, these drug substance particles would be released.

### BRIEF DESCRIPTION OF THE DRAWINGS

In the drawings:

FIG. 1 illustrates a cross section of one embodiment of an annular layer drug substance made using the nanolayer die.

FIG. 2 illustrates one embodiment of a product created by the nano die.

FIG. 3 illustrates another embodiment of a product created by the nano die.

FIG. 4 illustrates another embodiment of a product created by the nano die.

FIG. 5 illustrates a further embodiment of a product created by the nano die.

FIG. 6 illustrates a typical flow channel for a product created by the nano die.

FIGS. 7 illustrate examples of fold structures using a layer folding technique.

### DETAILED DESCRIPTION

General principles regarding the methods and the extrusion die may be found in United States Patent Publication No. 2012/0189789 “Method and Apparatus for Forming High Strength Products” and in U.S. Pat. No. 7,690,908 issued Apr. 6, 2010. Other methods are described in U.S. Pat. Nos. 6,669,458, 6,533,565 and 6,945,764. Each of the aforesaid publication or patent is herein incorporated by reference in its entirety.

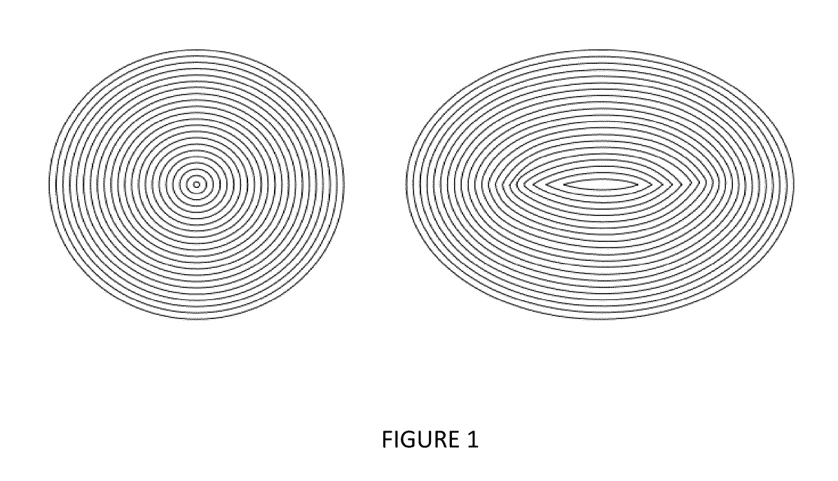

FIG. 1 illustrates a cross section of one embodiment of an annular layer drug substance made using the nanolayer die.

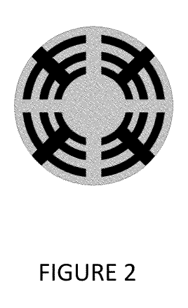

The nano die may also be used to create products which will have an increased interfacial surface area (see FIGS. 2-5). Sections of the layers mentioned above may be separated by ‘stems’ comprised of a single material or mixture. Each stem may be made of its own respective material or mixture allowing for the properties desired in that stem. A layer, stem or combination of the two may then be removed by some process, whether it is mechanical in nature such as peeling or chemical in nature such as dissolving. If one of the materials or mixtures used in the stem along with one or more of the materials used in the layers may all be removed, the result would be a core with stems protruding from the surface. These stems would have branches (layers) attached with a large surface area exposed to the environment. In the figure above, there are alternating layers of grey and black material separated by alternating grey and black stems. Only six layers are shown in each ‘stream’ for illustrative purposes but may comprise of thousands of layers. If all the black material were removed, the result would be a grey core with four stems each with six branches of material. This greatly increases the surface area exposed to the environment. By tailoring the rate at which the different materials dissolve along with the geometry, one could control the release rate of a drug substance by controlling the amount of surface area exposed to the environment. If the stems were to dissolve faster, a drug substance that broke up into sections could also be made.

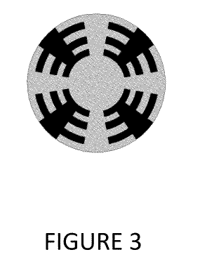

In the embodiment of FIG. 3, the stems are tapered radially inwards. The stems may also be made to be tapered radially outwards. The stems and branches may all be made to have different thicknesses and there may be any number of each.

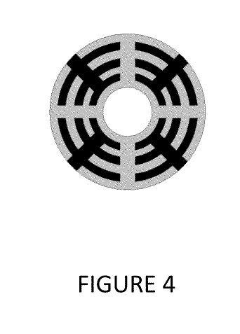

In the embodiment of FIG. 4, the core is comprised of a tube made of the grey material. Examples of a core include a solid rod, a hollow tube, a wire, or a profile all of which may either be coextruded or extruded onto and may be comprised of any materials with or without layers. The core may also be absent. An outer and/or inner layer may also be added and may be composed of multiple layers and may be comprised of any suitable material or materials.

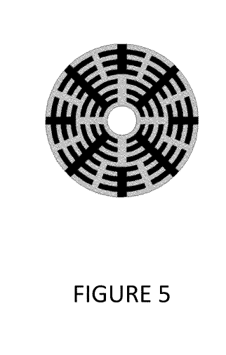

Multiple layers of streams and stems may also be used to be able to create geometries like the one pictured in FIG. 5. Theses layers may contain different numbers of layers, streams and stems in different orientations.

### Folding in a Coextrusion Die

Time released drug substances may also be made through a typical coextrusion head but with layers manipulated through folding to create additional layers. Such technology is described in U.S. Pat. No. 7,690,908 issued Apr. 6, 2010.

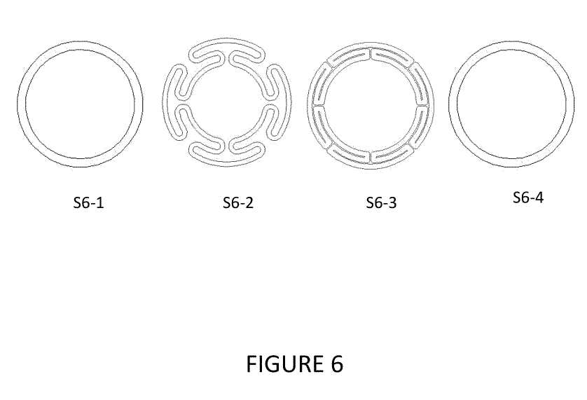

This approach to creating multilayered products begins with a typical flow channel for a product, as is illustrated in FIG. 6 (in the example of FIG. 6 the cross-section of this flow channel is an annular ring). The flow channel is then morphed to create folds in the flow channel (steps S6-1 to S6-3). These folds are oriented and propagated in such a way so that the flow may be converged back to a flow passage with a typical cross section but now with a multiplied number of layers (step S6-3 to S6-4). One advantage of this method of layer multiplication over others is that the layers remain continuous around the product.

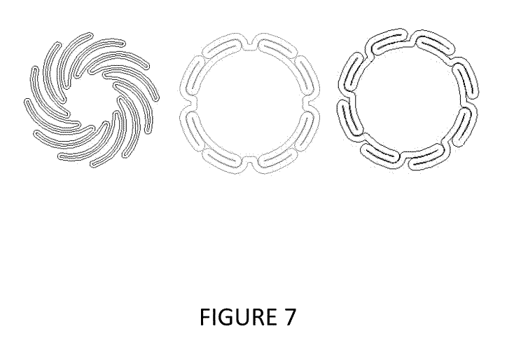

Some other examples of how the folds may be oriented are illustrated in FIG. 7.

The initial flow may contain any number of suitable materials in any number of layers and the layer multiplication process may be performed multiple times. The number of folds and the relative length that they stretch may also vary.

These layer geometries formed through this method allow for a way of controlling the time release of a drug substance much like the nano die.

### Stent

This aforesaid layer folding technique may also be used to create an expanding product such as a stent. A natural weakness at the interface of the folds or skin layer may be designed into a stem such that the stem can separate from the underlying support which may be dissolved either ex vivo or in vitro. The product so formed could break or seperate at this interface and expand into a larger shape. This expanding product could contain a drug substance and be used in such applications as a drug substance releasing stent.

1. A medical tubular device comprising: a polymeric tube containing nano or micro-sized features and a drug substance.

2. A medical tubular device according to claim 1 wherein said medical tubular device is a stent.

3. A medical tubular device comprising: a multi-component polymeric tube containing an embedded stem of a first polymer and a support surface surrounding said embedded stem containing a second polymer wherein each of said first and second polymer may contain one or more drug substances.

4. A medical tubular device according to claim 3 wherein said medical tubular device is an orally administrable medicament such as a pill or capsule.

5. A medical device comprising a polymer solid of annular rings emanating from the center of a drug substance core.

6. A medical device according to claim 5 wherein said device is a pill or capsule comprising a polymer solid of annular rings emanating from the center of a drug substance core.

7. A medical device comprising a polymer solid of annular rings containing a drug substance emanating from a center inactive core.
